(19) United States
(12) Patent Application Publication
(10) Pub. No.: US 2009/0191283 A1
(43) Pub. Date: Jul. 30, 2009

(54) TREATMENT OF HEADACHES, NECK PAIN, JOINT PAIN AND INFLAMMATORY-TYPE PAIN

(76) Inventors: Bryan Todd Oronsky, Los Altos Hills, CA (US); Neil Charles Oronsky, Los Altos Hills, CA (US)

Correspondence Address: MORRISON & FOERSTER LLP, 755 PAGE MILL RD, PALO ALTO, CA, 94304-1018, US

(21) Appl. No.: 12/019,460

(22) Filed: Jan. 24, 2008

(51) Int. Cl. A61K 31/21; A61K 33/04; A61K 31/405; A61K 31/381; A61P 25/00; A61K 31/343; A61K 31/34

(52) U.S. Cl. 424/702; 514/706; 514/470; 514/509; 514/415; 514/469; 514/443

(57) ABSTRACT

The formulation provides a method of treating headaches, neck, joint and inflammatory-type pain in a mammalian subject by administering to the subject a therapeutically effective amount of a nitrate-containing compound and a therapeutically effective amount of a selenium-containing compound. The formulation also provides kits and systems for practicing the subject methods.

### FIELD

The formulation is generally in the field of treatments of headache, neck and joint pain, and other types of inflammatory pain.

### BACKGROUND

Headaches is a term used to describe a chronic, recurring or occasional aching or pain that can be mild or severe enough to disrupt daily activities and occurs in one or more areas of the head, face, mouth, or neck. Most recurrent, episodic headaches are primary or benign (i.e., not caused by other medical conditions) and include tension-type, cluster, migraine headaches, chronic paroxysmal hemicrania, and hemicrania continua. New-onset, persistent headaches may have a secondary cause, due to various diseases and disorders such as tumor, infection, malignant hypertension, increased intracranial pressure, drugs, drug withdrawal, clot or aneurysm.

Headache is one of the most common human ailments, yet it continues to present significant challenges to understanding and treatment. Two of the most common headaches are migraines and tension headaches.

Migraine is a common, under-diagnosed, and under-treated neurological disorder. Although migraine is the most common cause of severe, recurring headache, headache is only one of the many ways the disease manifests itself. Migraine may also include visual disturbances, alterations in consciousness, photophobia, or phonophobia. The condition can be truly debilitating and the pain can interfere with a person's ability to live a normal productive life. Indeed, attacks can force the sufferer to abandon everyday activities for up to three days. Even in symptom-free periods, sufferers may live in fear of the next attack. Of the different types of migraines, classical migraine (migraine with aura) and common migraine (migraine without aura) are the two most prevalent.

Among the wide range of possible migraine triggers are emotional stress, intense physical exertion, weather changes, bright or flickering lights, high altitude, travel motion, and changes in sleep patterns. More than 100 foods have the capacity to trigger migraine headaches. Low blood sugar has also been known to trigger headaches, and fasting can often precipitate migraines. Likewise, chemicals, such as tyranine, phenylethylamine, tannin, sulfites, or monosodium glutamate, found in some foods, may trigger headache in some people. In children with migraines, common triggers are eating ice cream, anxiety, and fear.

Drugs that have been used in an attempt to treat migraine include: ergotamine and ergotamine-like agents, serotonin agonists, and caffeine with ergots or other pharmacologic agents (see e.g., Silberstein, S. D., Curr. Opinion Neurology 7:258-263 (1994); Welch, K. M. A., New Engl J. Med. 329:1476-1483 (1993); Dumar, K. L., J. Gen. Int. Med. 9:339-348 (1994); Saadah, H., Headache 32:95-97 (1992); and Becker, Arzneimittelforshung (42(4):552-555 (1992)). All of these drugs are thought to initially relieve migraine-associated pain by causing vasoconstriction. Unfortunately, this leads to numerous side effects such as chest pain or pressure, flushing, generalized tingling sensations, nausea, vomiting, pain in the legs and arms, asthenia, drowsiness, and dizziness. Acute ergotism is a particularly pernicious side effect of ergot drugs and is characterized by severe central and peripheral vasoconstriction, nausea, vomiting, diarrhea, colic, headache, vertigo, paresthesia, and possibly convulsive seizures.

Tension headaches are the most common type of headache. Tension headaches are distinguished from other headache syndromes largely on the basis of the quality, intensity, location, and duration of the pain. Typically, tension headaches last from 30 minutes to 7 days and both sides of the head are usually affected. Patients often describe the pain as a steady non-pulsing “vice-like” feeling of pressure without the accompanying symptoms of nausea, vomiting, photophobia and phonophobia that characterize migraines. The suspected etiology of a tension headache is muscle contraction with localized pain in the bitemporal, occipital, and nuchal regions of the head. Preventative medications can include daily antidepressants.

Representative treatments are described in U.S. Pat. Nos. 5,536,241, 6,258,032, 6,265,441, 6,284,794, and 6,649,605; U.S. Patent Application Nos. 2002/0072543, and 2004/0097562; and Lassen et al., Cephalalgia 18:27-32 (1998); Christiansen et al. Cepalalgia 19:661-667 (1999); Christiansen et al., Cepalalgia 20:437-444 (2000); Christiansen et al., Cepalalgia 20:445-454 (2000); and Lassen et al., Cepalalgia 23:877-886 (2003). While significant advances have been made in dealing with headaches specifically migraines, none has proven to be broadly effective for an extended time frame or helpful in prevention of reoccurrence of migraines.

It is therefore an object of the formulation to provide a method and composition for the treatment and prevention of migraine, tension and other types of headache.

### SUMMARY

Headaches are treated or prevented by administering to the subject a therapeutically effective amount of a nitrate-containing compound and a therapeutically effective amount of a selenium-containing compound. These can be provided separately, in combined formulations or in kits or combined packaging. In some embodiments, the selenium containing compound is an inorganic selenium (iSe) compound, such as inorganic selenite. In other embodiments, the selenium containing compound is an organic selenium (oSe) compound. In some embodiments, the organic nitrate containing compound is glycerol trinitrate, isosorbide-dinitrate, isobutyl nitrate, or isopentyl nitrate and isosorbide-5-mononitrate. In some embodiments, the method further comprises administering to the subject a therapeutically effective amount of an inhibitor of indoleamine-2,3-dioxygenase, such as D-tryptophan, 1-methyl-tryptophan, β-(3-benzofuranyl)-alanine, β-(3-benzo(b)thienyl)-alanine, or 6-nitro-D-tryptophan.

An individual with, or at risk of, a migraine headache, tension headache or headache due to a primary or secondary origin is treated by administration of a therapeutically effective amount of an organic nitrate containing compound and a therapeutically effective amount of a selenium-containing compound, to decreases the incidence or severity of the migraine in the subject. Recurrence of chronic migraine episodes can be prevented by administering to a subject a subanginal or anginal dose of an organic nitrate containing compound and a therapeutically effective amount of a selenium-containing compound, to prevent recurrence of chronic migraine episodes in the subject.

### DETAILED DESCRIPTION

### I. Definitions

“In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

The term “unit dosage form,” as used herein, refers to physically discrete units suitable as unitary dosages for human and animal subjects, each unit containing a predetermined quantity of compounds calculated in an amount sufficient to produce the desired effect in association with a pharmaceutically acceptable diluent, carrier or vehicle. The specifications for the unit dosage forms depend on the particular compound employed and the effect to be achieved, and the pharmacodynamics associated with each compound in the host.

The term “physiological conditions” is meant to encompass those conditions compatible with living cells, e.g., predominantly aqueous conditions of a temperature, pH, salinity, etc. that are compatible with living cells.

A “pharmaceutically acceptable excipient,” “pharmaceutically acceptable diluent,” “pharmaceutically acceptable carrier,” and “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, dileuent, carrier and adjuvant” as used in the specification and claims includes both one and more than one such excipient, dileuent, carrier, and adjuvant.

As used herein, a “pharmaceutical composition” is a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including, rectal, parenteral, intraperitoneal, intradermal, and intracheal.

As used herein, “pharmaceutically acceptable derivatives” of a subject compound includes salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs.

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not the environment of use.

By “substantially continuous” as used in, for example, the context of “substantially continuous subcutaneous infusion” or “substantially continuous delivery” is meant to refer to delivery of drug (e.g., nitrate-containing compound, an inorganic selenium-containing compound) in a manner that is substantially uninterrupted for a pre-selected period of drug delivery (other than a period associated with, for example, a bolus injection). Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

Unless defined otherwise, all technical and scientific terms used herein have the same meaning as commonly understood by one of ordinary skill in the art to which this invention belongs. Although any methods and materials similar or equivalent to those described herein can be used in the practice or testing, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a subject” includes a plurality of such subjects and reference to “the compound” includes reference to one or more compounds and equivalents thereof known to those skilled in the art, and so forth.

### II. Formulations

The formulation is based on the implication of nitric oxide (NO), a widespread endogenous vasodilator, in the pathophysiology of head pain, including migraine, through a variety of mechanisms which may involve extracerebral artery dilation and the direct activation of perivascular nerve fibers. In addition, the formulation is also based on the observation that, while intermittent exposure to organic nitrate-containing compounds can provoke headaches and spontaneous migraine attacks, continuous or repeated exposure to organic nitrate-containing compounds results in the development of nitrate tolerance, or tachyphylaxis, with an attenuation of the induced headache response.

Nitrate tolerance, or tachyphylaxis, has been attributed to several factors including the depletion of sulfhydryl groups, which are necessary co-factors for the biochemical conversion of the nitrate group to NO, and increased free radical production. Because NO can react with oxygen free radicals to form the powerful oxidant peroxynitrite (ONOO⁻), free radical generation may mediate nitrate tolerance by decreasing NO bioavailability. NO depletion in turn results in decreased adenylate cyclase activation and cyclic guanosine monophosphate (cGMP) production which is the final step in the pathway of vascular dilatation. Therefore, a decrease in NO formation results in a decrease in the production of cGMP, which results in a subsequent decrease or absence of the vasodilation which can result in headache.

Furthermore, a decrease in free radical formation from thiol-dependent reduction also results in depletion of NO. For example, the inorganic form of selenium, selenite (SeO3-2) undergoes thiol-dependent reduction to produce selenide (H2Se) which supplies selenium for the synthesis of selenoproteins. At lower concentrations, the major effects of selenite are related to its role as a micronutrient. However, at higher concentrations, the reaction between selenite and reduced thiols, such as glutathione (OSH), produces free radicals. In addition, selenite also depletes GSH levels by catalyzing the oxidation of GSH to GSSG, thereby acting as a pro-oxidant, since only GSH and not GSSG can reduce reactive oxygen species. Accordingly, the increased production of free radicals from the reaction between selenite and reduced thiols, such as glutathione, results in the depletion of NO. Moreover, because reduced glutathione is a potential donor of the SH group which is required for the formation of NO from organic nitrates, the oxidation of GSH to GSSG may further augment the induction of nitrate tolerance.

Furthermore, in instances in which the precipitant of the headache episode is depression or stress related (e.g., anxiety, anger, worry, excitement, shock, etc), the tachyphylaxis (nitrate tolerance) resulting from the nitrate and selenite administration can be combined with an inhibitor of the tryptophan catabolizer IDO (indoleamine-2,3-dioxygenase). Inhibition of IDO results in an increase in endogenous tryptophan levels. Since tryptophan is a precursor to serotonin, inhibition of the enzyme that catabolizes tryptophan, results in an increase in the level of serotonin. Therefore, inhibition of IDO potentiates the tachyphylaxis (nitrate tolerance) resulting from the nitrate and selenite administration.

A. Nitrates

Organic nitrate containing compound suitable for use in the formulation can be provided in a variety of forms. Examples of suitable Organic nitrate containing compounds include, but are note limited to, glycerol trinitrate (nitroglycerin), isosorbide-dinitrate, isosorbide-5-mononitrate, isobutyl nitrate, and isopentyl nitrate. In representative embodiments the organic nitrate containing compound is glycerol trinitrate (nitroglycerin), both because of its ready availability in a variety of forms; pill, patch, ointment, cream, spray, inhaler, etc., and because its pharmacology is so well known. The many nitroglycerine equivalents and substitutes, such as p.o. clonidine, Dynacirc (isradipine), hydrazine, or long acting nifedipine and others known to the art, can be used to replace or to supplement nitroglycerine.

B. Selenium-Containing Compounds

Selenium-containing compound suitable for use in the formulation can be provided in a variety of forms. Selenium may be present in elemental form or as inorganic or organic selenium compounds. It is also noted that selenium occurs in different valence forms. For example, selenium compounds occur in which the selenium has a +4 valance or a +6 valence, as the selenite and selenate ions, respectively. In certain embodiments, the selenium-containing compound is an inorganic selenium-containing compound, referred to herein as an “iSe compound”. It is to be understood, however, that the particular inorganic forms of selenium compounds set forth herein are not to be considered exclusive.

Among the inorganic selenite and selenate forms of interest for use in the formulation are the water soluble alkali metal salts thereof, and particularly, the sodium and potassium salts, sodium and potassium selenite and selenate. Of particular interest are the water soluble alkali metal salts of selenite, and particularly, the sodium and potassium salts, that is, sodium selenite and potassium selenite.

In other embodiments, the selenium containing compound is an organic selenium containing compound, which can be referred to as “organoselenium compounds” or “oSe compound”. Exemplary organoselenium compounds include selenium compounds of cysteine and methionine, as well as an organic selenium compound such as RSeH, RSeR, RSeR′, RSeSeR and RSeSeR′, wherein R and R′ are the same or different and each is an aliphatic residue containing at least one reactive group selected from the group consisting of aldehyde, amino, alcoholic, carboxylic, phosphate, sulfate, halogen or phenolic reactive groups and combinations thereof.

C. Inhibitors of IDO

Suitable inhibitors of the tryptophan-catabolizing enzyme IDO (indoleamine-2,3-dioxygenase) include, but are not limited to, D-trytophan, 1-methyl-tryptophan, β-(3-benzofuranyl)-alanine, β-(3-benzo(b)thienyl)-alanine, and 6-nitro-D-tryptophan. In representative embodiments, the inhibitor of IDO is not nitroglycerine.

D. Combination Therapy

For use in the subject methods, the subject compounds may be formulated with or otherwise administered in combination with other pharmaceutically active agents, including other compounds useful for treating migraines. The subject compounds may be used to provide an increase in the effectiveness of another chemical, such as a pharmaceutical, or a decrease in the amount of another chemical, such as a pharmaceutical, that is necessary to produce the desired biological effect.

For example, the subject methods can be combined with total histamine blockades, allergy shots against common migraine precipitants or food/environmental avoidance of common migraine triggers. Antimigraine drugs suitable for use in combination therapy are well-known and described in, for example, U.S. Pat. Nos. 4,650,810, 4,914,125, 4,916,125, 4,994,483, 5,021,428, 5,200,413, 5,242,949, 5,248,684, 5,273,759, 5,317,103, 5,364,863, 5,399,574, 5,434,154, 5,441,969, 5,464,864, 5,466,699, 5,468,768, 5,491,148 and 5,494,910. Anti-migraine drugs most commonly used in treatment of migraine fall into the following groups: ergot alkaloids, beta-blocking agents, calcium channel blocking agents, antidepressants, selective 5-HT₁ agonists (sumatriptan), sedatives, local anesthetics, adrenergic blocking agents and mixtures thereof.

The nitrate/selenium combination also can be administered in combination with vasoconstrictive agents such as phenylephrine hydrochloride (in SUDAFED®), ergotamine, a triptan such as sumatriptan (IMITREX®), prostaglandins, thromboxane A₂, leukotriene D₄, angiotensin II, vasopressin, neuropeptide Y, endothelin, nicotine, caffeine, catecholamines, norepinephrine, epinephrine, isoproterenol, dopamine, ephedrine, phenylisopropylamines, phenylephrine, amphetainine, metraminol, methoxamine, lysergic acid, lysergic acid diethylamine, tetrahydrozoline hydrochloride, tetrahydrozoline hydrochloride with zinc sulfate and other known vasoconstrictors.

The nitrate/selenium can also be combined with an antiemetic such as meclizine, ginger, Droperidol (INAPSINE®), Metoolopramide (REGLAN®), Ondansetron (ZOFRAN®), droperidone, metocloprainide, ondansetron, benzquinamide (EMETE-CON®), diphenidol (VONTROL®), prochlorperazine (COMPAZINE®), promethazine (PHENERGAN(®), serotonin receptor antagonists including granisetron (KYTRIL®), dolasetron (ANZEMET®), corticosteroids, dronabinol (MARINOL®), haloperidol (HALDOL®), chlorpromazine (THORAZINE®), perphenazine (TRILAFON®), trimethobenzamide hydrochloride (TICAN®).

The compounds described herein for use in combination therapy with subject compounds may be administered by the same route of administration (e.g. intrapulmonary, oral, enteral, etc.) that the compounds are administered. In the alternative, the compounds for use in combination therapy with the subject compounds may be administered by a different route of administration that the compounds are administered.

Kits with unit doses of organic nitrate-containing compound and the inorganic or organic selenium-containing compound are provided. In such kits, in addition to the sterile containers containing the unit doses will be an informational package insert describing the use and attendant benefits of the subject compounds in treating the pathological condition of interest.

Preferred compounds and unit doses are those described herein above. In some embodiments, the organic nitrate-containing compound is provided for topic administration, such as, for example, a transdermal patch. In some embodiments, the selenium-containing compound is provided for oral administration.

E. Pharmaceutical Preparations

The compounds can be incorporated into a variety of formulations for therapeutic administration by a variety. of routes. More particularly, the organic nitrate-containing compound and/or selenium-containing compound can be formulated into pharmaceutical compositions by combination with appropriate, pharmaceutically acceptable carriers, diluents, excipients and/or adjuvants, and may be formulated into preparations in solid, semi-solid, liquid or gaseous forms, such as tablets, capsules, powders, granules, ointments, solutions, suppositories, injections, inhalants and aerosols. The organic nitrate-containing compounds and inorganic or organic selenium-containing compound can be formulated together and administered to the subject at the same time or can be formulated separately and administered to the subject at the same time or at a different time.

Pharmaceutically acceptable excipients such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents, are readily available to the public.

Parenteral routes of administration other than inhalation administration include, but are not necessarily limited to, subcutaneous, intramuscular, intrathecal, intracapsular, intraspinal, intrasternal, and intravenous routes, i.e., any route of administration other than through the alimentary canal. Parenteral administration can be carried to effect systemic or local delivery of the agent. Where systemic delivery is desired, administration typically involves invasive or systemically absorbed topical or mucosal administration of pharmaceutical preparations.

The subject compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agent can also be delivered to the subject by enteral administration. Enteral routes of administration include, but are not necessarily limited to, oral and rectal (e.g., using a suppository) delivery.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985; Remington: The Science and Practice of Pharnacy, A. R. Gennaro, (2000) Lippincott, Williams & Wilkins. The composition or formulation to be administered will, in any event, contain a quantity of the agent adequate to achieve the desired state in the subject being treated.

Topical formulations can be in the form of a transdermal patch, ointment, paste, lotion, cream, or gel. Topical formulations may include one or more of a penetrating agent, thickener, diluent, emulsifier, dispersing aid, or binder. Where the compound is formulated for transdermal delivery, the compound may be formulated with or for use with a penetration enhancer. Penetration enhancers, which include chemical penetration enhancers and physical penetration enhancers, facilitate delivery of the compound through the skin, and may also be referred to as “permeation enhancers” interchangeably. Physical penetration enhancers include, for example, electrophoretic techniques such as iontophoresis, use of ultrasound (or “phonophoresis”). Chemical penetration enhancers are agents administered either prior to, with, or immediately following compound administration, which increase the permeability of the skin, particularly the stratum corneum, to provide for enhanced penetration of the drug through the skin.

Compounds that have been used to enhance skin permeability include: the sulfoxides dimethylsulfoxide (DMSO) and decylmethylsulfoxide (C10 MSO); ethers such as diethylene glycol monoethyl ether, dekaoxyethylene-oleylether, and diethylene glycol monomethyl ether; surfactants such as sodium laurate, sodium lauryl sulfate, cetyltrimethylainmonium bromide, benzalkonium chloride, Poloxamer (231, 182, 184), Tween® (20, 40, 60, 80) and lecithin; the 1-substituted azacycloheptan-2-ones, particularly 1-n-dodecylcyclazacycloheptan-2-one; alcohols such as ethanol, propanol, octanol, benzyl alcohol; petrolatums, such as petroleum jelly (petrolatum), mineral oil (liquid petrolatum); fatty acids such as C₈-C₂₂ and other fatty acids (e.g., isostearic acid, octanoic acid, oleic acid, lauric acid, valeric acid); C₈-C₂₂ fatty alcohols (e.g., oleyl alcohol, lauryl alcohol); lower alkyl esters of C₈-C₂₂ fatty acids and other fatty acids (e.g., ethyl oleate, isopropyl myristate, butyl stearate, methyl laurate, isopropyl myristate, isopropyl palmitate, methylpropionate, ethyl oleate); monoglycerides of C₈-C₂₂ fatty acids (e.g., glyceryl monolaurate); tetrahydrofurfuryl alcohol polyethylene glycol ether; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; di-lower alkyl esters of C₆-C₈ diacids (e.g., diisopropyl adipate); ethyl acetate; acetoacetic ester; polyols and esters thereof such as propylene glycol, ethylene glycol, glycerol, butanediol, polyethylene glycol, and polyethylene glycol monolaurate; amides and other nitrogenous compounds such as urea, dimethylacetamide (DMA), dimethylformamide (DMF), 2-pyrrolidone, N-alkylpyrrolidone, e.g., 1-methyl-2-pyrrolidone; ethanol amine, diethanol amine and triethanolamine; terpenes; alkanones, and organic acids, particularly salicylic acid and salicylates, citric acid and succinic acid. Additional chemical and physical penetration enhancers are described in, for example, Transdermal Delivery of Drugs, A. F. Kydonieus (ED) 1987 CRL Press; Percutaneous Penetration Enhancers, eds. Smith et al. (CRC Press, 1995); Lenneruas et al., J Pharm Pharmacol 2002; 54(4):499-508; Karande et al., Pharm Res 2002; 19(5):655-60; Vaddi et al., J Pharm Sci 2002 July; 91(7):1639-51; Ventura et al., J Drug Target 2001; 9(5):379-93; Shokri et al., Int J Pharm 2001; 228(1-2):99-107; Suzuki et al., Biol Pharm Bull 2001; 24(6):698-700; Alberti et al., J Control Release 2001; 71(3):319-27; Goldstein et al., Urology 2001; 57(2):301-5; Kiijavainen et al., Eur J Pharm Sci 2000; 10(2):97-102; and Tenjarla et al., Int J Pharm 1999; 192(2):147-58.

Where the compound is formulated with a chemical penetration enhancer, the penetration enhancer is selected for compatibility with the compound, and is present in an amount sufficient to facilitate delivery of the compound through the skin of a subject, e.g., for delivery of the compound to the systemic circulation.

In one embodiment, one or both of the compounds are provided in a drug delivery patch, e.g., a transmucosal or transdermal patch, and can be formulated with a penetration enhancer. The patch generally includes a backing layer, which is impermeable to the compound and other formulation components, a matrix in contact with one side of the backing layer, which matrix provides for sustained release, which may be controlled release, of the compound, and an adhesive layer, which is on the same side of the backing layer as the matrix. The matrix can be selected as is suitable for the route of administration, and can be, for example, and can be a polymeric or hydrogel matrix.

In one embodiment of particular interest, one or more of the compounds (e.g., nitrate-containing compound, selenium-containing compound, and IDO inhibitor) are administered in aerosol formulation via intrapulmonary inhalation. One or more of the compounds can be formulated into pressurized acceptable propellants such as dichlorodifluoromethane, propane, nitrogen.

Mechanical devices designed for intrapulmonary delivery of therapeutic products, include, but are not limited to, nebulizers, metered dose inhalers, and powder inhalers, all of which are familiar to those of skill in the art. Specific examples of commercially available devices suitable for the practice of this formulation are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; the SPINHALER® powder inhaler, manufactured by Fisons Corp., Bedford, Mass.; the “standing cloud” device of Inhale Therapeutic Systems, Inc., San Carlos, Calif.; the AIR inhaler manufactured by Alkermes, Cambridge, Mass.; and the AERx pulmonary drug delivery system manufactured by Aradigm Corporation, Hayward, Calif. Of particular interest are the PARI LC PLUS®, the PARI LC STAR®, and the PARI BABY™ nebulizers by PARI Respiratory Equipment, Inc., Monterey, Calif.

Formulations for use with a metered dose inhaler device may generally comprise a finely divided powder. This powder may be produced by lyophilizing and then milling a liquid conjugate formulation and may also contain a stabilizer such as human serum albumin (HSA). Typically, more than 0.5% (w/w) HSA is added. Additionally, one or more sugars or sugar alcohols may be added to the preparation if necessary. Examples include lactose, maltose, mannitol, sorbitol, sorbitose, trehalose, xylitol, and xylose. The amount added to the formulation can range from about 0.01 to 200% (w/w), preferably from approximately 1 to 50%, of the conjugate present. Such formulations may then lyophilized and milled to the desired particle size.

The properly sized particles may then suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,2-tetrafluoroethane, or combinations thereof. Suitable surfactants may include sorbitan trioleate and soya lecithin. Oleic acid may also be useful as a surfactant. This mixture may then be loaded into the delivery device. An example of a commercially available metered dose inhaler suitable for use in the formulation is the Ventolin metered dose inhaler, manufactured by Glaxo Inc., Research Triangle Park, N.C.

Formulations for powder inhalers may comprise a finely divided dry powder containing conjugate and may also include a bulking agent, such as lactose, sorbitol, sucrose, or mannitol in amounts which facilitate dispersal of the powder from the device, e.g., 50% to 90% by weight of the formulation. The particles of the powder may have aerodynamic properties in the lung corresponding to particles with a density of about 1 g/cm² having a median diameter less than 10 micrometers, preferably between 0.5 and 5 micrometers, most preferably of between 1.5 and 3.5 micrometers. Powders may be generated and/or delivered by methods disclosed in U.S. Pat. Nos. 5,997,848, 5,993,783, 5,985,248, 5,976574, 5,922,354, 5,785,049 and 5,654,007.

For oral preparations, the subject compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In certain embodiments, for oral preparations the tablets may be enteric coated or otherwise protected to ensure better survival of the pharmaceutically active compound (e.g., inorganic or organic selenium-containing compound) through the stomach. An enteric coating is a coating that prevents release of the active agent until the dosage form reaches the small intestine. Enteric-coated dosage forms comprise, for example an inorganic or organic selenium-containing compound coated with an enteric polymer. The enteric polymer is generally non-toxic and is predominantly soluble in the intestinal fluid, but substantially insoluble in the gastric juices. Examples include polyvinyl acetate phthalate (PVAP), hydroxypropylmethyl-cellulose acetate succinate (HPMCAS), cellulose acetate phthalate (CAP), methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate, cellulose acetate butyrate, cellulose acetate propionate, methacrylic acid/methacrylate polymer (acid number 300 to 330 and also known as EUDRAGIT L), which is an anionic copolymer based on methacrylate and available as a powder (also known as methacrylic acid copolymer, type A NF, methacrylic acid-methyl methacrylate copolymer, ethyl methacrylate-methylmethacrylate-chlorotrimethylammonium ethyl methacrylate copolymer, and combinations comprising one or more of the foregoing enteric polymers. Other examples include natural resins, such as shellac, SANDARAC, copal collophorium, and combinations comprising one or more of the foregoing polymers. Yet other examples of enteric polymers include synthetic resin bearing carboxyl groups. The methacrylic acid:acrylic acid ethyl ester 1:1 copolymer solid substance of the acrylic dispersion sold under the trade designation EUDRAGIT L-100-55™ may be suitable.

The subject compounds can be made into suppositories by mixing with a variety of bases such as emulsifying bases or water-soluble bases. The compounds can be administered rectally via a suppository. The suppository can include vehicles such as cocoa butter, carbowaxes and polyethylene glycols, which melt at body temperature, yet are solidified at room temperature.

### III. Methods

A. Patients to be Treated

Methods for preventing and/or treating headaches specifically, but not exclusively, tension headaches and migraine episodes, including chronic migraine episodes, by administering to a subject in a continuous fashion an effective amount of an organic nitrate containing compound and a therapeutically effective amount of a selenium-containing compound. In addition, chronic refractory migraine episodes can be treated prophylactically according to the subject methods with continuous subanginal or anginal dosages of an organic nitrate containing compound in combination with an iSe. In the case of acute migraine episodes, the dosage of the organic nitrate containing compound is increased to an anginal or supra-anginal level.

Furthermore, in instances in which the precipitant of the migraine episode or tension headache is depression or stress related (e.g., anxiety, anger, worry, excitement, shock, etc), the tachyphylaxis (nitrate tolerance) resulting from the nitrate and selenite administration can be combined with an inhibitor of the tryptophan catabolizer IDO (indoleamine-2,3-dioxygenase). Inhibition of IDO results in an increase in endogenous tryptophan levels. Since tryptophan is a precursor to serotonin, inhibition of the enzyme that catabolizes tryptophan, results in an increase in the level of serotonin.

Typically, the length of migraine is from about two hours to two days. Examples of causes of migraine include: stress related, e.g., anxiety, anger, worry, excitement, shock, depression, overexertion, changes of routine and changes of climate, food-related, e.g., chocolate, cheese and other dairy products, red wine, fried food and citrus fruits, sensory-related, e.g., bright lights or glare, loud noises and intense or penetrating smells, menstruation and contraceptive drugs.

Subjects suitable for treatment include individuals that have, or are at risk of, chronic migraine episodes that may or may not be associated with aura. In a migraine attack, most but not all sufferers first experience early warning symptoms which signal the commencement of a migraine episode. All the senses can be affected, but not all sufferers experience all forms of sensory deviation. This phase is thought to occur as the blood supply to the brain is restricted. Symptoms of a migraine attack include, but are not limited to: (1) vision distortion, such as sparkling colored zigzag “halo” which distorts the vision, patches of distortion within the field of vision (somewhat akin to looking through a rain spattered pane of glass), development of acute sensitivity to light, or other less common visual symptoms; (2) distortion of taste, such as experiencing a “sweet taste” upon the palate in the absence of a sweet compound, such as sugar; (3) distortion in hearing, such as experiencing tinnitus (e.g., ringing of the ears) or a certain loss of sound definition, or acute sensitivity to sound; (4) sensitivity to touch, such as experiencing a “tingling” sensations often in the face and extremities, some lose feeling altogether (e.g., parasthesia, temporary paralysis); and (5) other symptoms, such as dizziness, loss of balance, nausea, vomiting, sugar craving, stomach pain, and sudden fatigue or sudden irritability with no apparent cause. Once the attack enters the second stage, all of the early warning symptoms can develop further, almost always accompanied by extreme head pain. This second stage can have a duration of a few hours or several days.

The formulations are useful not only for the treatment of headaches; as shown in the examples, the formulation are useful in patients with joint pain or pain in the entheses due to inflammatory type arthritis which can be broken down into a few different classifications: (1) Rheumatoid arthritis. (2) Seronegative spondyloarthropathies characterized by inflammation at the insertion of tendons to bone, which is referred to as enthesitis. This group includes ankylosing spondylitis, reactive arthritis or Reiter's syndrome, psoriatic, juvenile spondylitis and enteropathic arthropathy in inflammatory bowel disease among others. The common thread running through many of these syndromes is the presence of the histocompatibility antigen HLA-B27. 3) Crystal-induced arthritis which includes gout and calcium pyrophosphate deposition disease or pseudogout. 4) Other inflammatory arthopathies which include systemic lupus erythematosis (SLE), sarcoidosis, polymyositis and dermatomyositis, polymyalgia rheumatica, Lyme disease. The composition are also useful for other conditions such as scleroderma

Nitroglycerin and selenite have also alleviated cervicogenic pain in patients with all different types of neck conditions such as spondylosis, cervical radiculopathy, torticollis, chronic cervical strain, cervical facet syndromes, and cervical stenosis and in one patient with diffuse chronic muscle spasm due to cerebral palsy.

B. Methods of Administration

The following methods of administration are put forth so as to provide those of ordinary skill in the art with a complete disclosure and description of how to make and use the formulation, and is not intended to limit the scope of what the inventors regard as their invention nor are they intended to represent that the experiment below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

C. Dosage Forms

In pharmaceutical dosage forms, the organic nitrate-containing compound and the inorganic or organic selenium-containing compound may be administered in the form of a pharmaceutically acceptable salt, or may also be used alone or in appropriate association, as well as in combination, with other pharmaceutically active compounds. The following methods and excipients are merely exemplary and are in no way limiting.

The compounds can be administered to a host using any available conventional methods and routes suitable for delivery of conventional drugs, including systemic or localized routes. In general, routes of administration contemplated by the formulation include, but are not necessarily limited to, enteral or parenteral routes, such as intrapulmonary or intranasal delivery.

Conventional and pharmaceutically acceptable routes of administration include intramuscular, intrathecal, intratracheal, subcutaneous, intradermal, intravenous, transdermal, rectal, nasal, oral and other parenteral routes of administration. Routes of administration may be combined, if desired, or adjusted depending upon the agent and/or the desired effect. For example, the nitrate-containing compound may be administered by a topical route (e.g., transdermal route) and the inorganic or organic selenium-containing compound may be administered by a different route, such as oral route or intravenous route. The compositions can be administered in a single dose or in multiple doses.

In one embodiment, the organic nitrate-containing compound is administer by topical administration (e.g., by transdermal administration).

Depending on the subject and the administration route, the organic nitrate-containing compound, the inorganic or organic selenium-containing compound, and the IDO inhibitor may each be administered in dosages of, for example, 0.1 μg to 10 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus, for example, oral dosages may be about ten times the injection dose. Higher doses may be used for localized routes of delivery.

In some embodiments, the dosage of organic nitrate-containing compound administered to the subject is selected in order to provide for a subanginal dose of a organic nitrate-containing compound, including about 0.1 mg/hr to about 0.3 mg/hr of organic nitrate-containing compound, including about 0.11 mg/hr to about 0.29 mg/hr, about 0.12 mg/hr to about 0.28 mg/hr, about 0.13 mg/hr to about 0.27 mg/hr, about 0.14 mg/hr to about 0.26 mg/hr, about 0.15 mg/hr to about 0.25 mg/hr, about 0.16 mg/hr to about 0.24 mg/hr, about 0.17 mg/hr to about 0.23 mg/hr, about 0.18 mg/hr to about 0.22 mg/hr, about 0.19 mg/hr to about 0.21 mg/hr, about 0.20 mg/hr of organic nitrate-containing compound.

In representative embodiments, the organic nitrate-containing compound will be nitroglycerin and the administration route will be topical, e.g., transdermal administration. An exemplary transdermal delivery system for nitroglycerin is the NITRO-DUR™ patch, such as the 10 cm² NITRO-DUR™ patch comprising 40 mg of nitroglycerin or the 2.5 cm² NITRO-DUR™ patch comprising 10 mg of nitroglycerin. The 10 cm² NITRO-DUR™ patch can be cut in half to provide two 5 cm² NITRO-DUR™ patches each comprising 20 mg of nitroglycerin. In other embodiments, the nitroglycerin is provided in a 2% ointment for topical application as cream or rub. The ointment can be applied to a 0.5 inch² area once a day to provide for delivery of 20 mg of nitroglycerin. The ointment can be applied to a 0.25 inch² area once a day to provide for delivery of 10 mg of nitroglycerin.

In some embodiments, the dosage of inorganic or organic selenium-containing compound administered to the subject is selected in order to provide for a dose of a organic nitrate-containing compound, including about 1 μM/kg to about 20 μM/kg, about 2 μM/kg to about 19 μM/kg, about 3 μM/kg to about 18 μM/kg, about 4 μM/kg to about 17 μM/kg, about 5 μM/kg to about 16 μM/kg, about 6 μM/kg to about 15 μM/kg, about 7 μM/kg to about 14 μM/kg, about 8 m μM/kg to about 13 μM/kg, about 9 μM/kg to about 12 μM/kg, about 10 μM/kg to about 11 μM/kg.

Typically, the duration of the administration of organic nitrate-containing compound and inorganic or organic selenium-containing compound will be continual and range from about 1 week to about 6 months or more, including about 22 weeks, about 20 weeks, about 18, weeks, about 16 weeks, about 15 weeks, about 14 weeks, about 13 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, etc. In some embodiments, the duration of the administration of the subject compounds may be more, such as more than about 6, months, including about 7 moths or more, about 8 months or more, about 9 months or more, about 10 months or more, about 1 year or more, about 18 months or more, etc.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

Although the dosage used will vary depending on the clinical goals to be achieved, a suitable dosage range is one which provides up to about 1 μg to about 1,000 μg or about 10,000 μg of the subject composition to provide for a desired effect in a subject animal.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds. Similarly, unit dosage forms for injection or intravenous administration may comprise the compound (s) in a composition as a solution in sterile water, normal saline or another pharmaceutically acceptable carrier.

The following protocols can be used to optimize dosage administration.

(i) Constant Dose of Selenite and Varying Dose of Nitroglycerine for Treatment of Migraines and Tension Headaches

Organic nitrate-containing compound and the inorganic or organic selenium-containing compound are administered to subjects. Subjects suitable for treatment include individuals without organic disease that have, or are at risk of, chronic migraine episodes that may or may not be associated with aura as defined by the International Headache Society (IHS-2003 1.1 migraine without aura, 1.2 migraine with aura). Subjects will also be included if they have an IHS probable migraine and/or tension-type headache.

A constant dose of selenite will be administered to potentiate the nitrate tachyphylaxis at a dose of about 200 μg/day. The organic nitrate-containing compound is nitroglycerin and the administration route is by continual transdermal dose-rising administration dose starting at 6.6 mg/hr (obtained by cutting a 10 cm2 NITRO-DUR™ patch into eight strips of 1.25 cm² each) and titrating upwards gradually until the minimal dose which produces the desired effect of sustained headache relief or headache-free state via nitrate tachyphylaxis is reached. The maximal allowable dose will be a 10 cm² NITRO-DUR™ patch comprising 40 mg of nitroglycerin.

The effectiveness of the therapy will be determined by providing the subjects with a headache diary which chronicles the attack frequency, severity, duration and associated nausea/vomiting for a certain time period prior to administration of selenite and nitroglycerin in order to establish a “baseline” of parameters prior to the onset of treatment. These measures will be compared to attack frequency, severity, duration and frequency of associated nausea/vomiting after administration of selenite and nitroglycerin.

Quantification of these measures will be attempted by use of a descriptive scale that will include interrogatories to the subjects requesting characterization of the migraine pain as (i) none, (ii) mild, (iv) moderate, or (v) severe. Moreover, a descriptive categorical scale will be used to characterize the subject's headache-related disability. On this scale, subjects describe their disability as:
(1) none;
(2) mildly impaired (e.g., having a little bit of difficulty doing what I usually do);
(3) moderately impaired (e.g., having a great deal of difficulty doing what I usually do and can only do very minor activities); or
(4) severely impaired (e.g., requiring bed rest).

An 11-point verbal numerical rating scale for pain will be another measurement method of determining the effectiveness of the treatment. On this scale, the subjects are asked to describe their pain as a number between zero and ten, with zero being no pain and ten being the worst pain imaginable. Subjects will also have to complete a series of psychiatric rating scales to assess the effects of the continual nitrate therapy on mood and cognition. Headache characteristics (e.g., severity of pain, location, and quality), its aggravation by physical activity and the presence or absence of accompanying symptoms, including nausea, vomiting, phono-phobia, photo-phobia, etc., will be simultaneously recorded and compared with the diagnostic criteria for migraines of the International Headache Society.

The primary outcome will be the percentage of subjects who achieve and maintain a “headache pain free” state. Other secondary outcomes will include: (1) rate of sustained headache relief (e.g., moderate or severe pain becoming and maintaining a level of mild or none); (2) the amount of time to change from a baseline (e.g., in the absence of the treatment) in terms of hours or days; (3) reduction in headache-free disability (e.g., moderate or severe changing to and/or maintaining a level of mild or none); and (4) change from baseline (e.g., in the absence of the treatment) of associated symptoms.

(ii) Constant Dose of Nitroglycerine and Varying Dose of selenite for Treatment of Migraines and Tension Headaches.

Organic nitrate-containing compound and the inorganic or organic selenium-containing compound are administered to subjects. Subjects suitable for treatment include individuals without organic disease that have, or are at risk of, chronic migraine episodes that may or may not be associated with aura as defined by the International Headache Society (IHS-2003 1.1 migraine without aura, 1.2 migraine with aura). Subjects will also be included if they have an IHS probable migraine and/or tension-type headache.

A constant dose of the organic nitrate containing compound nitroglycerin will be administered transdermally in a continual dose of about 10 mg/hr. The selenium-containing compound will be administered to potentiate the nitrate tachyphylaxis in a oral dose-rising administration starting at about 100 μg and increasing gradually until the minimal dose which produces the desired effect of sustained headache relief or headache-free state via nitrate tachyphylaxis is reached. The maximal allowable dose will be about 400 μg of selenite.

The effectiveness of the therapy will be determined by providing the subjects with a headache diary which chronicles the attack frequency, severity, duration and associated nausea/vomiting for a certain time period prior to administration of selenite and nitroglycerin in order to establish a “baseline” of parameters prior to the onset of treatment. These measures will be compared to attack frequency, severity, duration and frequency of associated nausea/vomiting after administration of selenite and nitroglycerin. Quantification of these measures will be as described in (i) above.

(iii) Varying Dose of Selenite and Varying Dose of Nitroglycerine and Varying Dose of an IDO Inhibitor for Treatment of Migraines and Tension Headaches.

Organic nitrate-containing compound and the inorganic or organic selenium-containing compound are administered to subjects. Subjects suitable for treatment include individuals without organic disease that have, or are at risk of, chronic migraine episodes that may or may not be associated with aura as defined by the International Headache Society (IHS-2003 1.1 migraine without aura, 1.2 migraine with aura) and that may by precipitated by stress and anxiety. Subjects will also be included if they have an IHS probable migraine and/or tension-type headache.

The organic nitrate-containing compound is nitroglycerin and the administration route is by continual transdermal dose-rising administration starting at about 6.6 mg/hr (obtained by cutting a 10 cm² NITRO-DUR™ patch into eight strips of 1.25 cm² each) and titrating upwards gradually until the minimal dose which produces the desired effect of sustained headache relief or headache-free state via nitrate tachyphylaxis is reached. The maximal allowable dose will be a 10 cm² NITRO-DUR™ patch comprising 40 mg of nitroglycerin.

The selenium-containing compound selenite will be administered to potentiate the nitrate tachyphylaxis in a oral dose-rising administration starting at about 100 μg and increasing gradually until the minimal dose which produces the desired effect of sustained headache relief or headache-free state via nitrate tachyphylaxis is reached. The maximal allowable dose will be about 400 μg of selenite.

The IDO inhibitor compound D-tryptophan will be administered in conjunction with the nitrate tachyphylaxis and to increase the level serotonin. D-tryptophan will be administered in an oral dose-rising administration starting at about 1 g and increasing gradually until the minimal dose which produces the desired effect of sustained headache relief or headache-free state is reached. The maximal allowable dose will be about 3 g of D-tryptophan.

The effectiveness of the therapy will be determined by providing the subjects with a headache diary which chronicles the attack frequency, severity, duration and associated nausea/vomiting for a certain time period prior to administration of selenite, nitroglycerin, and D-tryptophan in order to establish a “baseline” of parameters prior to the onset of treatment. These measures will be compared to attack frequency, severity, duration and frequency of associated nausea/vomiting after administration of selenite, nitroglycerin, and D-tryptophan. Quantification of these measures will be as described in (i) above.

The present invention will be further understood by reference to the following non-limiting examples.

### EXAMPLES

### Example 1

### Treatment of Patients with Headaches

(I) Treatment of Patients with Migraine Headaches

(a) A female patient presented with a migraine headache with an intensity of 9-10/10. The migraines which occurred at a frequency of 3-4×/week had previously been treated with narcotics.

Treatment: ½ of a 0.1 mg/hr nitroglycerin patch applied 2×/day (every 12 hours) initially to ankle then to shoulder+2 pills of 250 mg sodium selenite/day (Twin Labs)

Response to treatment: Has only had 1 migraine in a 1 month period

Side effects of treatment: Constant crippling headache for 4 days which was partially alleviated by SUDAFED

(b) A female patient presented with a migraine headache with an intensity of 10/10. The migraines occurred at a frequency of 2×/week and had previously been treated with narcotics, NTHEs, acetaminophen.

Treatment: 0.1 mg/hr nitroglycerin patch applied 2×/day (every 12 hours) initially to ankle then to shoulder+2 pills of 250 mg sodium selenite/day (Twin Labs)

Response to treatment: Has only had 1 migraine in a 2 month period

Side effects of treatment: Severe headache for 5 days which was partially alleviated by SUDAFED

(c) A female patient presented with a migraine/tension headache with an intensity of 7-9/10. The headache frequency was everyday and had previously been treated with narcotics, acetaminophen, NTHEs.

Treatment: 0.1 mg/hr nitroglycerin patch applied 2×/day (every 12 hours) to shoulder+2 pills of 250 mg sodium selenite/day (Twin Labs)

Response to treatment: Had 1 month of daily 2/10 headaches for about 1 month but then increased the selenite pills from 2 to 3/day and now the headaches are completely gone.

Side effects of treatment: Debilitating headache for 6 days which was not alleviated by SUDAFED.

(d) A male patient presented with an every other day migraine headache with an intensity of 10/10, and the patient stated “The headaches were so bad that I was unable to work or enjoy my life.” Prior treatment included extensive workup by neurologists. Tried narcotics, Topamax, beta blockers, calcium channel blockers, corticosteroids, triptans, trigger point injections all without success

Treatment: 0.6 mg/hr nitroglycerin patch applied 2×/day (every 12 hours) to ankle for 1 week then shoulder+2 pills of 250 mg sodium selenite/day (Twin Labs)

Response to treatment: Had intermittent headaches for about 1 month. He remained headache free for 2 months. At this point he was advised to stop the treatment and has remained headache free off treatment for 1 month.

Side effects of treatment: Debilitating headache for 4 days which was not alleviated by SUDAFED.

(e) A female patient presented with a 3-4×/wk migraine headache, with an intensity of 10/10. Prior treatment included narcotics, Tylenol, NTHEs

Treatment: 0.1 mg/hr nitroglycerin patch applied 1×/day to shoulder+2 pills of 250 mg sodium selenite/day (Twin Labs).

Response to treatment: headaches improved to 2/10

Side effects of treatment: extreme fatigue. Patient has since dropped out of the study due to fatigue.

(f) A female patient presented with a 1-2×/month migraine headache, with an intensity of 10/10. Prior treatment included narcotics, Tylenol, NTHEs, triptans

Treatment: 0.2 mg/hr nitroglycerin patch applied 2×/day to shoulder+3 pills of 250 mg sodium selenite/day (Twin Labs)

Response to treatment: headaches have disappeared. In fact, patient stopped treatment and has continued to remain headache-free for over 1 month.

Side effects of treatment: worsened headaches for 5 days partially alleviated by SUDAFED.

(g) A female patient presented with a 3-4×/wk migraine headache with an intensity of 10/10. Prior treatment included narcotics, Tylenol, NTHEs, trigger point injections. Patient was treated with 0.4 mg/hr nitroglycerin patch applied 2×/day to shoulder+2 pills of 250 mg sodium selenite/day (Twin Labs). Headaches and neck pain have disappeared, patient reported she is sleeping much better and there were no side effects

(h) A female patient presented with a once every other month migrains headache with an intensity of 10/10. Prior treatment included narcotics, Tylenol, NTHEs, trigger point injections. Patient was treated with 0.2 mg/hr nitroglycerin patch applied 1×/day to shoulder+2 pills of 250 mg sodium selenite/day (Twin Labs). Headaches have disappeared.

Side effects of treatment: worsened headaches for 12 days partially alleviated by SUDAFED.

(II) Treatment of Patients with Cervicogenic Tension Headaches

(a) A female patient presented with cervicogenic tension headache with “severe” neck pain with an intensity of 7 or 8/10. The frequency of headaches was everyday and prior treatment included narcotics, NTHEs, Tylenol.

Treatment: 0.4 mg/hr nitroglycerin patch initially applied 1×/day to shoulder+2 pills of 250 mg sodium selenite/day (Twin Labs)

Response to treatment: Had 2 straight weeks of “murderous” headaches unalleviated by SUDAFED then daily headaches and neck pain tapered off in intensity to 3-4/10 for about 1 month. At this point patient was instructed to apply the nitroglycerin patch 2×/day (every 12 hours) and the headaches and neck pain disappeared

Side effects of treatment: 2 straight weeks of “murderous” headaches unalleviated by SUDAFED

(b) A female patient presented with cervicogenic headache with an intensity of 3-4×/wk. Prior treatment included narcotics, Tylenol, NTHEs. Patient was treated with 0.2 mg/hr nitroglycerin patch applied 1×/day applied to shoulder+2 pills of 250 mg sodium selenite/day (Twin Labs)

Response to treatment: Started in October. Had 1 day of a 2/10 headache in 4 months.

Side effects of treatment: None

(c) A male patient presented with migraine/cervicogenic headache with a frequency of everyday and an intensity of 7/10. Prior treatment included narcotics, NTHEs, acetaminophen, morphine cream.

Treatment: 0.4 mg/hr nitroglycerin patch applied 1×/day applied to shoulder+2 pills of 250 mg sodium selenite/day (initially) then 0.1 mg/hr nitroglycerin patch applied 1×/day applied to ankle+2 pills of 250 mg sodium selenite/day

Response to treatment: Became so sick initially with headaches, nausea and vomiting that he had to stop treatment. Restarted 1 month later with lower dose of patch applied to ankle. Now he has daily headaches 4/10 in intensity.

Side effects of treatment: See above paragraph

(d) A female patient presented with a daily cervicogenic tension headache, with an intensity of 10/10. Prior treatment included narcotics, Tylenol, NTHEs.

Treatment: 0.2 mg/hr nitroglycerin patch applied 1×/day to shoulder+3 pills of 250 mg sodium selenite/day (Twin Labs)

Response to treatment: headaches decreased to 2-3/10

Side effects of treatment: extreme headache for 1.5 weeks. Then diffuse swelling over body which caused her to drop out of the study

(e) A male patient presented with a daily cervicogenic tension headache, with an intensity of 10/10. Prior treatment included narcotics, Tylenol, NTHEs, trigger point injections

Treatment: 0.2 mg/hr nitroglycerin patch applied 1×/day to shoulder+2 pills of 250 mg sodium selenite/day (Twin Labs)

Response to treatment: none

Side effects of treatment: worsened headaches

(f) A female patient presented with a daily cervicogenic tension headache, with an intensity of 7-8/10. Prior treatment included narcotics, Tylenol, NTHEs, trigger point injections

Treatment: 0.2 mg/hr nitroglycerin patch applied 1×/day to shoulder+2 pills of 250 mg sodium selenite/day (Twin Labs)

Response to treatment: headaches are gone and neck feels much more mobile

Side effects of treatment: worsened headaches for 4 days alleviated by SUDAFED

(g) A female patient presented with a daily cervicogenic tension headache, with an intensity of 9-10/10. Prior treatment included narcotics, Tylenol, NTHEs, trigger point injections, BoTox which worked well for 3 months but is too expensive

Treatment: 0.2 mg/hr nitroglycerin patch applied 2×/day to shoulder+3 pills of 250 mg sodium selenite/day (Twin Labs).

Response to treatment: headaches and neck pain have virtually disappeared. Patient was still pain free after a month of stopping treatment.

Side effects of treatment: worsened headaches partially alleviated by SUDAFED for 1.5 weeks.

(h) A female patient presented with a daily cervicogenic tension headache with an intensity of 5/10. Prior treatment included narcotics, Tylenol, NTHEs, trigger point injections.

Treatment: 0.2 mg/hr nitroglycerin patch applied 1×/day to shoulder+2 pills of 250 mg sodium selenite/day (Twin Labs)

Response to treatment: headaches have virtually disappeared

Side effects of treatment: worsened headaches for 13 days

(i) A male patient presented with a daily cervicogenic tension headache, with an intensity of 7-8/10. Prior treatment included narcotics, Tylenol, NTHEs, trigger point injections

Treatment: 0.2 mg/hr nitroglycerin patch applied 1×/day to shoulder+2 pills of 250 mg sodium selenite/day (Twin Labs)

Response to treatment: headaches have virtually disappeared as well as neck pain

Side effects of treatment: worsened headaches totally alleviated by SUDAFED.

### Example 2

### Treatment of Patient with Inflammatory Arthropathies

(a) A female patient presented with Rheumatoid Arthritis with a pain level of 9-10/10 and the following symptoms: morning stiffness, diffuse polyarticular involvement with joint deformities, malaise, weakness, myalgias. Prior treatment included narcotics, antiinflammatories, corticosteroids, disease-modifying anti-rheumatologic drugs (DMARDs), immunosuppressants daily

Treatment: ½ of a 0.1 mg/hr nitroglycerin patch applied 2×/day (every 12 hours) initially to ankle then to shoulder+2 pills of 250 mg sodium selenite/day (Twin Labs)

Response to treatment: pain decreased to 5/10. Then started 0.3 mg/hr patch 2×/day with 3 selenite pills and pain levels have decreased to 1 or 2. Patient notices most effect in the hands, wrists, elbows and ankles

Side effects of treatment: Constant crippling headache for 6 days which was partially alleviated by SUDAFED

(b) A female patient presented with Rheumatoid Arthritis with pain levels of 10/10 and the following symptoms: morning stiffness, diffuse polyarticular involvement, malaise, weakness, myalgias

Prior treatment included narcotics, antiinflammatories, corticosteroids daily.

Side effects of treatment: Vertebral fractures and osteonecrosis from Prednisone

Treatment: 0.1 mg/hr nitroglycerin patch applied 2×/day (every 12 hours) to shoulder+2 pills of 250 mg sodium selenite/day (Twin Labs)

Response to treatment: pain decreased to 2/10. Able to move around and perform household chores for the first time in several years. “This treatment is better than Prednisone.”

Side effects of treatment: Constant crippling headache for 3 days which was partially alleviated by SUDAFED

(c) A male patient presented with a 10/10 pain level from Dermatomyositis, with the following symptoms: morning stiffness, diffuse polyarticular involvement, malaise, proximal muscle weakness, muscle tenderness

Prior treatment included narcotics, antiinflammatories, corticosteroids, disease-modifying anti-rheumatologic drugs (DMARDs) daily. Patient initially had an excellent response to Prednisone but later suffered steroid-induced osteoporotic rib and wrist fractures.

Treatment: 0.3 mg/hr nitroglycerin patch applied 2×/day (every 12 hours) to shoulder+3 pills of 250 mg sodium selenite/day (Twin Labs)

Response to treatment: pain decreased to 3-4/10. Pain from sacroilitis is unabated but most noticeable effect is in small joints of hands, ankles, wrists and elbows. Less morning stiffness and malaise. States that pain relief is better than with Prednisone

Side effects of treatment: Constant crippling headache for 2 weeks which was partially alleviated by SUDAFED

(d) A male patient presented with rheumatoid arthritis with pain levels of 10/10 and the following symptoms: diffuse joint pain, malaise, morning stiffness

Prior treatment included narcotics, anti-inflammatories, corticosteroids, disease-modifying anti-rheumatologic drugs (DMARDs) taken daily. Now only taking narcotics because he developed infections from immunosuppressive meds

Treatment: 0.3 mg/hr nitroglycerin patch applied 2×/day (every 12 hours) to shoulder+2 pills of 250 mg sodium selenite/day (Twin Labs)

Response to treatment: pain decreased to 5/10. Pain from sacroilitis is unabated or even worsened but most noticeable effect is in small joints of hands and knees. Considerably less morning stiffness and malaise.

Side effects of treatment: Constant crippling headache for 1 week which was partially alleviated by SUDAFED

(e) A female patient presented with rheumatoid arthritis with 10/10 pain levels and the following symptoms: morning stiffness, diffuse polyarticular involvement, malaise, weakness, myalgias

Prior treatment included narcotics, antiinflammatories, daily corticosteroids which she stopped due to exacerbation of her diabetes

Treatment: 0.1 mg/hr nitroglycerin patch applied 2×/day (every 12 hours) to shoulder+3 pills of 250 mg sodium selenite/day (Twin Labs)

Response to treatment: pain decreased to 3/10. Much more mobile. Sacroilitis, however, has not improved

Side effects of treatment: Constant headache for 7 days which was partially alleviated by SUDAFED.

(f) A patient presented with daily rheumatoid arthritis with pain levels of about 4-5/10 on average. Prior treatment included Prednisone. The combination gave him a very severe headache for 7 days, which subsequently improved to about 2/10 and he has started to taper off his Prednisone.

### Example 3

### Additional Treatments if Patient with Pain

(I) Treatment of Patient with Torticollis

A male patient presented with torticollis (wry painful neck but no headaches) with an intensity: 7-8/10. Prior treatment included narcotics, Tylenol, NTHEs, trigger point injections. Patient was treated with 0.2 mg/hr nitroglycerin patch applied 1×/day to shoulder+2 pills of 250 mg sodium selenite/day (Twin Labs). Neck felt 20% looser with no side effects.

(II) Treatment of Patient with Hemicrania Continua

A male patient presented with daily hemicrania continua with an intensity of 8-9/10. Prior treatment included narcotics, Tylenol, NTHEs, trigger point injections, calcium channel blockers, beta blockers, oxygen, triptans all without success. Patient was treated with 0.2 mg/hr nitroglycerin patch applied 2×/day to shoulder+2 pills of 250 mg sodium selenite/day (Twin Labs). Response to treatment: headaches are gone

Side effects of treatment: worsened headaches for 4 days.

(III) Treatment of Patient with Pseudotumor Cerebri

A male patient presented with daily pseudotumor cerebri and morning headache from sleep apnea, with an intensity of 10/10. Prior treatment included narcotics, Tylenol, NTHEs, trigger point injections, Topamax, triptans, blood pressure meds, Diamox, CPAP all without much success. Patient was treated with 0.2 mg/hr nitroglycerin patch applied 2×/day to shoulder+3 pills of 250 mg sodium selenite/day (Twin Labs)

Response to treatment: headaches are now 1/10 in intensity. Patient remained pain free after 2 months of treatment and was still pain free 3 weeks after discontinuing treatment.

Side effects of treatment: worsened headaches for 3 days improved with Imitrex.

(IV) Treatment of Patient with Cerebral Palsy

A patient presented with spastic quadriplegic cerebral palsy with daily pain levels around 6/10. The combination gave him a “horrendous” headache for 7 days but subsequently reported decreased pain and improved range of motion with about 2/10 pain levels

The preceding merely illustrates the principles. It will be appreciated that those skilled in the art will be able to devise various arrangements which, although not explicitly described or shown herein, embody the principles and are included within its spirit and scope. Furthermore, all examples and conditional language recited herein are principally intended to aid the reader in understanding the principles and the concepts contributed by the inventors to furthering the art, and are to be construed as being without limitation to such specifically recited examples and conditions.

We claim:

1. A method of treating a subject suffering from or susceptible to acute or chronic headaches whether primary or secondary, acute or benign, comprising:
administering to a subject suffering from or susceptible to headaches a therapeutically effective amount of an organic nitrate containing compound and a therapeutically effective amount of a selenium-containing compound,
wherein the administering decreases the incidence or severity of a headache in the subject.

2. The method of claim 1, wherein the selenium-containing compound is an inorganic selenium (iSe) containing compound.

3. The method of claim 2, wherein the iSe compound is inorganic selenite.

4. The method of claim 1, wherein the selenium-containing compound is an organic selenium (oSe) containing compound.

5. The method of claim 1, wherein the organic nitrate containing compound is glycerol trinitrate, isosorbide-dinitrate, isobutyl nitrate, isopentyl nitrate, or isosorbide-5-mononitrate.

6. The method of claim 1, wherein the method further comprises administering to the subject a therapeutically effective amount of an inhibitor of indoleamine-2,3-dioxygenase.

7. The method of claim 6, wherein the inhibitor of indoleamine-2,3-dioxygenase is D-tryptophan, 1-methyl-tryptophan, β-(3-benzofuranyl)-alanine, β-(3-benzo(b)thienyl)-alanine, or 6-nitro-D-tryptophan.

8. The method of claim 1 further comprising administering an emetic.

9. The method of claim 1 further comprising administering a vasoconstrictor.

10. A method for preventing recurrence of chronic headache episodes, comprising:
administering to a subject a subanginal or anginal dose of a organic nitrate containing compound and a therapeutically effective amount of a selenium-containing compound,
wherein the administering prevents recurrence of chronic headache episodes in the subject.

11. The method of claim 10, wherein the selenium-containing compound is an inorganic selenium (iSe) containing compound.

12. The method of claim 10, wherein the iSe compound is inorganic selenite.

13. The method of claim 10, wherein the selenium-containing compound is an organic selenium (oSe) containing compound.

14. The method of claim 10, wherein the organic nitrate containing compound is glycerol trinitrate, isosorbide-dinitrate, isobutyl nitrate, isopentyl nitrate, or isosorbide-5-mononitrate.

15. The method of claim 10, wherein the method further comprises administering to the subject a therapeutically effective amount of an inhibitor of indoleamine-2,3-dioxygenase.

16. The method of claim 15, wherein the inhibitor of indoleamine-2,3-dioxygenase is D-tryptophan, 1-methyl-tryptophan, β-(3-benzofuranyl)-alanine, β-(3-benzo(b)thienyl)-alanine, or 6-nitro-D-tryptophan.

17. The method of claim 10 further comprising administering an emetic or vasoconstrictor.

18. A method of treating a subject suffering from or susceptible to neck pain or joint pain comprising:
administering to a subject a subanginal or anginal dose of a organic nitrate containing compound and a therapeutically effective amount of a selenium-containing compound,
wherein the administering decreases the incidence or severity of neck pain in the subject.

19. The method of claim 18, wherein the selenium-containing compound is an inorganic selenium (iSe) containing compound.

20. The method of claim 18, wherein the iSe compound is inorganic selenite.

21. The method of claim 18, wherein the selenium-containing compound is an organic selenium (oSe) containing compound.

22. The method of claim 18, wherein the organic nitrate containing compound is glycerol trinitrate, isosorbide-dinitrate, isobutyl nitrate, isopentyl nitrate, or isosorbide-5-mononitrate.

23. The method of claim 18, wherein the method further comprises administering to the subject a therapeutically effective amount of an inhibitor of indoleamine-2,3-dioxygenase.

24. The method of claim 23, wherein the inhibitor of indoleamine-2,3-dioxygenase is D-tryptophan, 1-methyl-tryptophan, β-(3-benzofuranyl)-alanine, β-(3-benzo(b)thienyl)-alanine, or 6-nitro-D-tryptophan.
